(19) United States
(12) Patent Application Publication
(10) Pub. No.: US 2008/0148418 A1
(43) Pub. Date: Jun. 19, 2008

(54) Novel Zebrafish

(76) Inventors: Shiue-Lian Lin, Taipei (TW); Yin-Chun Chen, Taipei (TW); I-Teng Hung, Taipei (TW)

Correspondence Address: WPAT, PC;INTELLECTUAL PROPERTY ATTORNEYS, 2030 MAIN STREET, SUITE 1300, IRVINE, CA, 92614, US

(21) Appl. No.: 12/036,082

(22) Filed: Feb. 22, 2008

Related U.S. Application Data
(62) Division of application No. 11/314,725, filed on Dec. 22, 2005.

(51) Int. Cl. A01K 67/027; A01K 67/02

(52) U.S. Cl. 800/9; 800/21; 800/20

(57) ABSTRACT

This invention relates to novel zebrafish and preparation method thereof. This invention also relates to a novel animal model.

### CROSS-REFERENCE TO RELATED APPLICATIONS

This application claims priority to, and is a Divisional of, U.S. patent application Ser. No. 11/314, 725, filed on Dec. 22, 2005, now pending, which is hereby incorporated by reference in its entirety.

Although incorporated by reference in its entirety, no arguments or disclaimers made in the parent application apply to this divisional application. Any disclaimer that may have occurred during the prosecution of the above-referenced application(s) is hereby expressly rescinded. Consequently, the Patent Office is asked to review the new set of claims in view of all of the prior art of record and any search that the Office deems appropriate.

### FIELD OF THE INVENTION

This invention relates to novel zebrafish and preparation method thereof. This invention also relates to a novel animal model.

### DESCRIPTION OF PRIOR ART

Admiring and culturing ornamental fish is an interesting leisure activity for modern people. Gazing and feeding fish kept in aquaria offers people an incredible opportunity to enter an entirely different world—an exotic marine world under water. Varieties of aquatic plants, sands, gravels, rocks, and together with some other decorations compose scenes that can be splendid, fantasy, creative and soothing. Some backgrounds of aquarium are designed as majestic mountains with bushy forests. Others are designed as fantasic gardens full of clumps of aquatic plants which have tendrils and vines creeping outward and resemble bizarre flowers and herbs. With aid of light effect, ornamental fish show various shapes and images to provide people with a charming feeling. There are a lot of spots such as popular sightseeing resorts, commercial centers, hotels, and places providing such ornamental fish. A lot of family raise various set up aquaria decorated with various pet fishes, like Goldfish, Angelfish, different species of tropical fish, and marine fish in living room or gardens to convey a sense of loveliness and serenity.

Ornamental fish refers to those fish with bright color or unique shapes. These fish frequently are distributed all over the world and have a tremendous diversity. Some of fish live in fresh or salt water, some of them are from temperate or tropical zone. Some of ornamental fish are known as bright colors or bizarre shapes. Some of them are known as uncommon or precious species. In international ornamental fish markets, ornamental fish are generally classified as temperate zone freshwater fish, tropical zone freshwater fish or tropical zone saltwater fish.

Tropical freshwater ornamental fish are majority from rivers and lakes. With a tremendous diversity of species, these fish from these areas have diversely extraordinary shapes and distinct sizes, astonishing colors and pleasing beauty. According to the habit of these fish, most of them are from three areas. The first area is the countries in the regions of Amazon River in South America, such as Columbia, Paraguay, Guiana, Brazil, Argentina, and Mexico. The second one is the countries in South East Asia, such as Thailand, Malaysia, India, and Srilanka. And the third one is the regions nearby African Great Lakes, such as Lake of Malawi, Lake of Victoria and Lake of Tangonyika.

There are three well known groups of tropical freshwater ornamental fish: Neon Tetra, Angelfish, and Arowana. Neon Tetra such as Traffic Light Fish, Head-and-taillight Tetra, Needle Fish, Cardinal Neon Tetra, and Black Neon Tetra are small, vigorous, ravishing and brightly colored. Some species are translucent and are popular in the market of aquarium fish. Angelfish such as Red Rainbow Angelfish, Blue Rainbow Angelfish, Blue Band Green Rainbow Angelfish, Black Angelfish, Sesame Angelfish, Duck Angelfish, and Ruby Eye Diamond Angelfish are also splendidly beautiful in shapes and coloration. Unlike Neon Tetra, Angelfish swims in a temperate, casual and elegant way, resembling deities in the ancient history. Arowana such as Silver Arowana, Red Arowana, Golden Arowana, and Black Arowana is the third group, also known as “Living Fossil”. Fishes in this group swim gracefully, and are generally peaceful. They are expensive and popular in aquarium fish trade.

Ornamental fish belongs to entertainment fish industry and is one part of commercial fisheries and of economic importance. The demand in the market of ornamental fish is usually great throughout the world. Fish strains, particularly those artificial generated or those with special shapes and marvelous beauty, immediately attract most of customers. Therefore, the commerce in ornamental fish increases a great deal thereafter.

In the scientific classification, zebrafish is classified in order Cypriniformes, family Cyprinidae, and genus Danio. It originally comes from South Asia and is one of the most common species of tropical fish. Zebrafish has a slim torpedo-shape and horizontal blue and silver stripes at the side of the body. It grows to about 3 to 4 centimeters in overall length, and is considered hardy fish, being easily to be kept without sensitiveness of water condition. A zebrafish turns into sexually mature in about 3 months. A mature female zebrafish is able to give a spawning every few days. Zebrafish eggs are fertilized and develop outside of mother body. These transparent embryonic eggs grow not only synchronously but also rapidly, and the best range of temperature for developing embryonic eggs is between 25□ and 31□.

The mechanisms of the embryonic development of zebrafish are similar to those of mammals. Further, the large number of eggs of every spawning, zebrafish eggs has additionally other traits that make them the best tool for scientific research. These traits include, the transparency of embryonic eggs, fertilization outside of mother bodies, and short incubation time that a young fish, which swims and is able to search for food by itself, can be generated within 48 to 72 hours. Moreover, the eggs can be observed easily with the help of microscopes; organs inside the embryos such as nerves, muscle, heart, bloodstreams and blood cells become conspicuous when magnified. Melatonin occurs as early as 24 hours after fertilization. Basic body coloration and blue and silver stripes are gradually appeared. Currently, zebrafish is an important animal model widely used for scientific research in all kinds of field.

### BRIEF DESCRIPTION OF THE DRAWINGS

FIG. 1 shows an image of a mutant white zebrafish.

FIG. 2 shows images of green and red fluorescent zebrafish. (A) A male white zebrafish with green fluorescence. (B) A female white zebrafish with green fluorescence. (C) A male white zebrafish with red fluorescence. (D) A female white zebrafish with red fluorescentce.

FIG. 3 shows an image of a mutant transparent zebrafish.

FIG. 4 shows the process of generating transparent zebrafish. F0 parent is red fluorescent zebrafish. F1 progenies are red-golden zebrafish. F2 progenies are red-golden zebrafish (left panel) and transparent zebrafish (right panel).

### SUMMARY OF THE INVENTION

This invention provides a method for generating novel zebrafish, comprising: (a) breeding transgenic fluorescent zebrafish by self-hybridization; and (b) screening the new transgenic progenies showing different phenotype or behavior pattern from their parents.

This invention also provides a white zebrafish, a transparent zebrafish, and a red-golden zebrafish form the method of the present invention.

This invention further provides an animal model, which comprises a white zebrafish or a transparent zebrafish as an experimental animal.

### DETAILED DESCRIPTION OF THE INVENTION

This invention provides a method for generating novel zebrafish, comprising: (a) breeding transgenic fluorescent zebrafish by self-hybridization; and (b) screening the new transgenic progenies showing different phenotype or pattern from their parents.

The fluorescent zebrafish used in this invention is produced by transforming gene fragment containing inverted terminal repeats (ITR-R) of adeno-associated virus, actin gene promoter, fluorescent gene, SV40 poly A and inverted terminal repeats (ITR-L) of adeno-associated virus from upstream to downstream. The gene fragment is shown as below:

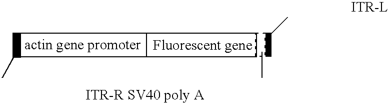

wherein the ITR is from the terminal repeat sequence of adeno-associate virus. In a preferred embodiment, the alpha-actin gene promoter of golden zebrafish and the red fluorescent gene are selected to construct the gene fragment. This gene fragment is transformed into golden zebrafish to prepare fluorescent zebrafish.

In the method of the present invention, the phenotype is selected from the group consisting of colors, fluorescent colors, body shapes, body sizes, body transparent levels, grain colors, stripe colors, fin shapes, fin sizes, tail shape, tail sizes, eye color, eye shapes; and any observable phenotypic differences from those of fluorescent mate.

The method of the present invention further comprises breeding the progenies of step (b) by self-hybridization to generate progenies.

The method of the present invention further comprises breeding the progenies of step (b) with fish with different phenotype or behavior pattern to generate progenies.

In a preferred embodiment, the fluorescent zebrafish is red fluorescent zebrafish. In a preferred embodiment, the novel zebrafish selected from the progenies is white zebrafish, wherein the white zebrafish does not express fluorescent gene. In a more preferred embodiment, the white zebrafish is used to generate pure strain of white zebrafish by self-hybridization. The pure strain can stabilize the expression of phenotype and pattern of white zebrafish. The white zebrafish of the present invention is characterized to snow white in whole body except eyes.

In a preferred embodiment, the novel zebrafish selected from the progenies is red-golden zebrafish, such red-golden zebrafish does not express fluorescent gene. In a more preferred embodiment, the red-golden zebrafish is used to generate transparent zebrafish by self-hybridization, such transparent zebrafish of the present invention is characterized to directly observe viscera by eyes.

The method of this invention can further comprise introducing fluorescent gene into the white zebrafish. Because the white zebrafish is snow white color in whole body and different with wild type zebrafish expressing deep blue and silver vertical streaks on the body, the introduced fluorescent gene or pigment gene in white zebrafish can significantly and effectively express special body color. In an embodiment, the fluorescent gene is selected from the group consisting of green fluorescent gene, red fluorescent gene, yellow fluorescent gene, orange fluorescent gene, cyan fluorescent gene and blue fluorescent gene. In a preferred embodiment, the fluorescent gene is green fluorescent gene. In a preferred embodiment, the fluorescent gene is red fluorescent gene.

The present invention also provides a white zebrafish, which is produced as described above. The white zebrafish is characterized to express snow white in whole body except eyes and has small and thin scales. In an embodiment, the white zebrafish of the present invention expresses snow white in whole body with small and thin scales, silver and white streak from vertebra to abdomen and black eyes.

The white zebrafish of the present invention can be used to generate novel zebrafish. In an embodiment, the novel zebrafish is prepared by transforming gene. The thin scales and pure white color of this novel zebrafish are helpful in expression of a transformed gene. The transformed gene, for example but not limiting, is fluorescent gene, pigment gene, body color relative gene or other gene related to patterns or phenotypes expressed on the surface of fish body. In a preferred embodiment, the novel zebrafish is prepared by transforming fluorescent gene wherein the fluorescent gene is selected from the group consisting of green fluorescent gene, red fluorescent gene, yellow fluorescent gene, orange fluorescent gene, cyan fluorescent gene and blue fluorescent gene. In a more preferred embodiment, the novel zebrafish is red fluorescent white zebrafish, which is prepared by transforming red fluorescent gene into white zebrafish. In a more preferred embodiment, the novel zebrafish is green fluorescent white zebrafish, which is prepared by transforming green fluorescent gene into white zebrafish.

In another embodiment, the novel zebrafish is prepared by mating with fish with different phenotype or behavior pattern. The thin scales and pure white color of this novel zebrafish are helpful in expression of variant phenotypes. For this reason, the white zebrafish can used to prepare new zebrafish different with parents by mating with fish having different phenotype, for example but not limiting, fluorescent zebrafish, fluorescent golden zebrafish, fluorescent zebra Leopard, golden zebrafish, little zebra, Leopard Danio, or other zebrafish.

The present invention also provides a transparent zebrafish, which is produced as described above, which is characterized to express transparent in whole body and directly observe by eyes. The transparent zebrafish can be further characterized to show transparent phenotype expression in whole life, from childhood to adult. The transparent phenotype does not disappear when growing. In a preferred embodiment, this zebrafish is transparent in whole body except black eyes, and the body color is transparent orange-yellow color from the original color of skeletal muscle of zebrafish. The viscera of the zebrafish can be observed directly by eyes.

The transparent zebrafish of the present invention can be used to prepare novel zebrafish. In an embodiment, the novel zebrafish is prepared by mating with fish with different phenotype or behavior pattern. In another embodiment, the novel zebrafish is prepared by transgenic technology.

The colorless expression of transparent zebrafish of the present invention is helpful to transformed gene expression, wherein the transformed gene includes fluorescent gene, pigment gene, body color relative gene, the gene related to phenotype on body surface, and, especially the transformed gene expressing inside the fish body.

In an embodiment, the novel zebrafish is prepared by transforming fluorescent gene. In a preferred embodiment, the transformed gene is fluorescent gene selected from the groups consisting of green fluorescent gene, red fluorescent gene, yellow fluorescent gene, orange fluorescent gene, cyan fluorescent gene and blue fluorescent gene. In a more preferred embodiment, the novel zebrafish is green-golden zebrafish prepared by introducing green fluorescent gene into the transparent zebrafish.

For the transparent body color, the novel zebrafish is prepared from transparent zebrafish by mating with fish with different phenotype or pattern, for example but not limiting, fluorescent zebrafish, fluorescent golden zebrafish, fluorescent zebra Leopard, golden zebrafish, little zebra, leopard Danio, or other zebrafish. In a preferred embodiment, the novel zebrafish is purple-golden zebrafish prepared by mating the transparent zebrafish with purple zebrafish.

This invention also provides a red-golden zebrafish, which is produced as described above, which does not express fluorescence.

In addition to ornamental use, the zebrafish of the present invention can be widely applied to medical research or other investigation of biological field, such as cellular signaling or embryo development research.

The present invention further provides an animal model, which comprises a white zebrafish or a transparent zebrafish as an experimental animal. The animal model is used in disease model, aging investigation, drug screening, toxicant screening, embryo development or organ differentiation. In an embodiment, the white zebrafish and transparent zebrafish can be prepared by the method described above.

To described detail, the animal model of the present invention is used in visual system model (Bilotta et al., International Journal of Developmental Neuroscience, 2001, Vol. 19(7):621-629), laboratory model in comparative study (Metscher et al., Developmental Biology, 1999, Vol. 210(1):1-14), immunological model system (Yoder et al., Microbes and Infection, 2002, Vol. 4(14):1469-1478), drug screening and validation (Sumanas et al., Drug Discovery Today Targets, 2004, Vol. 3(3):89-96), GATA transcription factor model (Heicklen-Klein et al., Seminars in Cell and Developmental Biology, 2005, Vol. 16(1):95-106), developmental model system, toxicological and transgenic research (Lele et al., Biotechnology Advances, 1996, Vol. 14(1):57-72; Huuskonen et al., Toxicology and Applied Pharmacology, 2005, Vol 207(2):495-500), developmental expression of thrombin (Jagadeeswaran et al., Blood Cells, Molecules, and Diseases, 1997, Vol. 23(2):147-156), pharmacological animal model (Goldsmith, Current Opinion in Pharmacology, 2004, Vol. 4(5):504-512), host model system of microbial pathology (Miller et al., Acta Tropica, 2004, Vol. 91(1):53-68), neurotoxicological model (Linney et al., Neurotoxicol and Teratol, 2004, Vol. 26(6):709-718), model for myelopoiesis during embryogenesis (Berman et al., Experimental Hematology, 2005, Vol. 33(9):997-1006), human disease model (Dooley et al., Curr Opin Genetics & Develop, 2000, Vol. 10(3):252-256) or genetic research model system (Dlugos et al., Pharmacol Biochem Behav, 2003, Vol. 74(2):471-480).

In the animal model of the present invention, the white zebrafish is used in phenotype of body color or pigment investigation, for example but not limiting, melanoma investigation.

In the animal model of this invention, transparent zebrafish is used in observation of viscera, such as heart, kidney, stomach, intestine, or liver. For the special property, the transparent zebrafish can be applied to research, for example but not limiting, heart disease including heart failure, arrhythmias or congenital heart disease, kidney disease including polycystic kidney disease or kidney failure, gastrointestinal disease including colon cancer, dysotility, malabsorption or diabetes, or liver disease including cirrhosis, liver damage or liver necrosis caused by alcohol, infection or toxin.

The animal model of the present invention comprises a white zebrafish or a transparent zebrafish as an experimental animal. In particular, the white zebrafish or the transparent zebrafish can be used for performing foreign gene transformation or gene mutation.

### EXAMPLE

The examples below are non-limiting and are merely representative of various aspects and features of the present invention.

Materials: Red fluorescent zebrafish (TK-2 Fluorescent zebra-Red, Product name: TK-2 Red Fluorescent Elf) was acquired from Taikong Corp. Wile-type strain of golden zebrafish was used for genetic manipulation. After introduction of gene encoding coral red fluorescent protein (RFP) into the golden zebrafish, the transgenic fish were capable of emitting red fluorescence from skeletal muscle and have golden vertical stripes on the body surface.

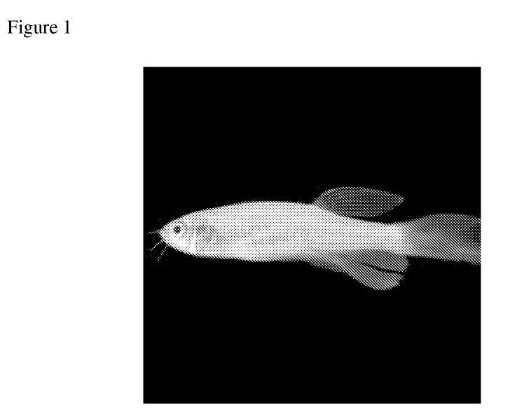

### Example 1 Preparation of White Zebrafish

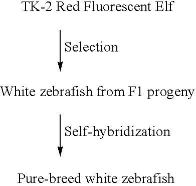 [0044] 1 Male and female red fluorescent zebrafish were crossbred to generate F1 progeny.
2 White zebrafish with a phenotype of nearly white skin without expression of fluorescent gene were selected from F1 progeny.
3 Selected male and female F1 white zebrafish were crossbred. White zebrafish of F2 progeny were selected again and self-hybridized for further one to two generations.
4 Pure-breed white zebrafish with stable expression of the phenotype were selected and kept. Pure-breed white zebrafish had snow-white skin, tiny and thin scales, white spots scattered below the spine, black eyes, and a maximal length of 6 centimeters (FIG. 1). FIG. 1 showed an image of a male white zebrafish with a long slim shape. The female white zebrafish was generally larger in size and had well-stacked belly. There are no other apparent characteristics to distinguish female white zebrafish from male one.

### Example 2 Preparation of Fluorescent Zebrafish from White Zebrafish

### (A) Methods

1. Collection of fertilized white zebrafish embryos: zebrafish were collected and isolated by separated net in tanks at 11 pm before dark cycle beginning. Eggs were collected every 15-20 minutes in the next morning while light cycle beginning. Around 30-40 eggs could be subjected to injection each time. In one experiment, about 250-300 embryos were injected.
2. Preparation of plasmid DNA: Plasmid DNA containing fluorescent genes was digested by restriction enzyme. Small scale of digested plasmid DNA was subjected to agarose gel electrophoresis for the assurance that all plasmid DNA (8.1 kb) had been linearized. For cytoplasmic microinjection, linearized plasmid DNA was extracted with phenol: chloroform (1:1), precipitated with ethanol, dried out, and dissolved in PBS with a concentration of 10 μg/ml.
3. Microinjection: Linearized plasmid DNA was mixed with phenol red containing 5×PBS and the final concentration was adjusted. Zebrafish microinjector (Drummond) was loaded with prepared plasmid DNA and a 10 μm diameter micropipette was used for injection. Approximately 2.3 nl of plasmid DNA was injected into the animal pole of each cell-stage embryo.
4. Culture of fertilized embryos: Microinjected fertilized embryos were washed with distilled water and incubated at 28.5□. After 24 hours, embryos were observed under fluorescent microscope for the expression of green fluorescence. Only those with the capability of emitting green fluorescence were kept.

### (B) Preparation of Green Fluorescent Zebrafish

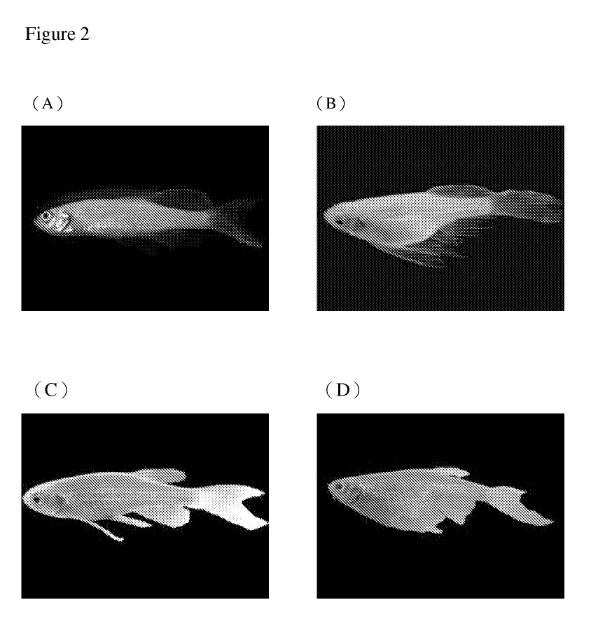

Plasmid pα-EGFPITR (Taikong Corp., Chou et al., Transgenic Research, 2001, 10: 303-315), containing green fluorescent gene, was linearized with Not I restriction enzyme. The following preparation steps were the same like that written in method (A).
FIG. 2 showed images of fluorescent zebrafish. FIG. 2 (A) showed an image of a male white zebrafish with green fluorescence, while FIG. 2 (B) showed an image of a female white zebrafish with green fluorescence.
(C) Preparation of red fluorescent zebrafish Plasmid p α-DsRedITR (Taikong Corp., TW 227735B), containing red fluorescent gene, was linearized with Not I restriction enzyme. The following preparation steps were the same like that written in method (A).
FIG. 2 showed images of fluorescent zebrafish. FIG. 2 (C) showed an image of a male white zebrafish with red fluorescence, while FIG. 2 (D) showed an image of a female white zebrafish with red fluorescence.
FIG. 2 clearly showed that fluorescent zebrafish derived from white zebrafish expressed fluorescent genes on the whole body. White zebrafish was different from wild-type strain which had dark-blue stripes on the body; therefore, white zebrafish was suitable to be used to generate fluorescent zebrafish which were capable of exerting apparent, even and good fluorescence.

### Example 3 Preparation of Transparent Zebrafish

1 Male and female red fluorescent zebrafish were crossbred to generate F1 progeny.
2 Red-golden zebrafish without expression of the fluorescent gene were selected from F1 progeny.
3 Selected male and female red-golden zebrafish from F1 progeny were crossbred. The produced progeny were selected and self-hybridized for further one to two generations. Only offsprings with transparent bodies were selected.
4 Only pure-breed transparent zebrafish with stable expression of the phenotype were selected and kept.

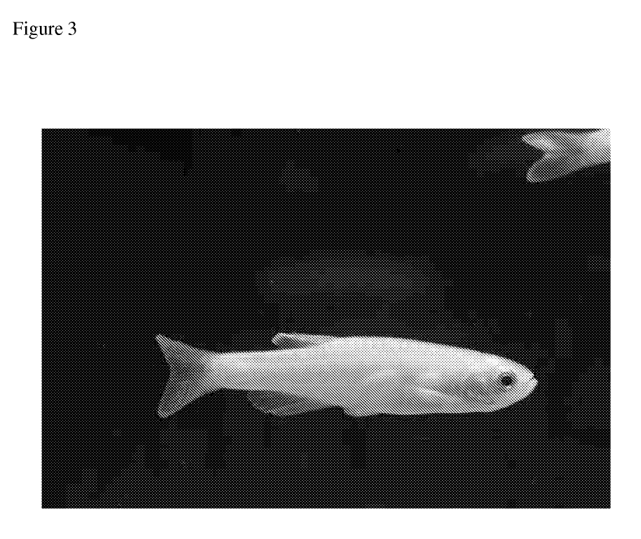

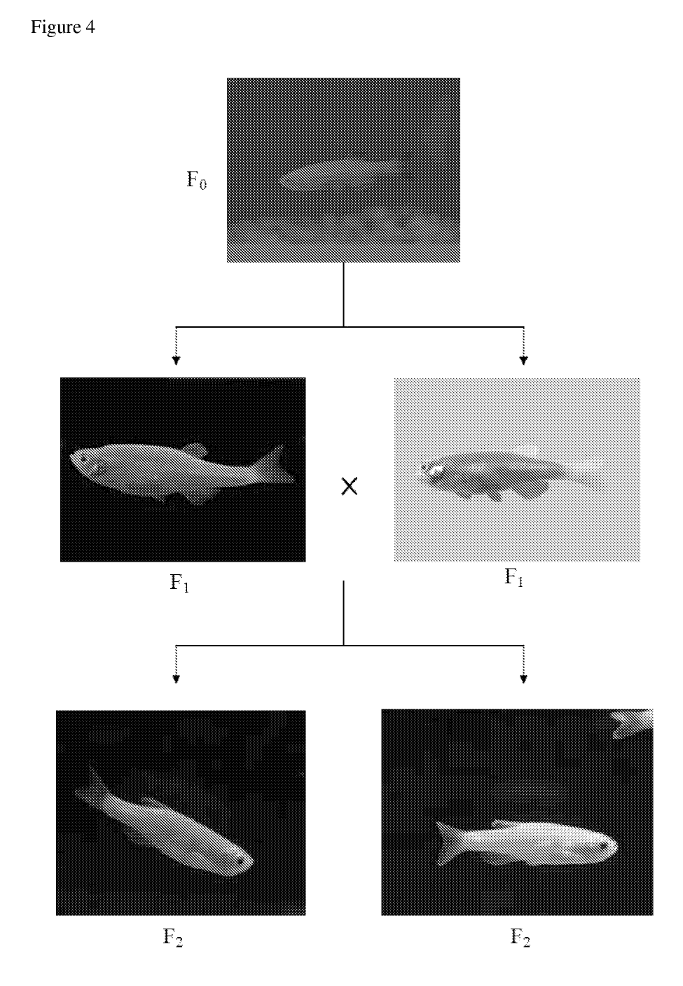

FIG. 4 showed the preparation process of transparent zebrafish. FIG. 3 showed an image of a transparent zebrafish. Except of the black eyes, whole fish expressed yellowish skin, and the viscera were directly observed. The maximal length of the fish was 6 centimeters.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The embryos, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

What is claimed is:

1. A method for generating a zebrafish, comprising:
(a) breeding transgenic fluorescent zebrafish by self-hybridization; and
(b) screening the new transgenic progenies showing different phenotype or behavior pattern from their parents.

2. A white zebrafish, which is produced as claimed in claim 1.

3. The white zebrafish of claim 2, which is characterized by snow white in whole body except eyes.

4. The white zebrafish of claim 2, which has small and thin scales.

5. The white zebrafish of claim 4, which is used to generate new kind of zebrafish.

6. The white zebrafish of claim 5, wherein the zebrafish is prepared by transforming fluorescent gene.

7. The white zebrafish of claim 6, wherein the fluorescent gene is selected from the group consisting of green fluorescent gene, red fluorescent gene, yellow fluorescent gene, orange fluorescent gene, cyan fluorescent gene and blue fluorescent gene.

8. The white zebrafish of claim 6, wherein the zebrafish is red or green fluorescence.

9. The white zebrafish of claim 5, wherein the novel zebrafish is prepared by mating with fish with different phenotype or behavior pattern.

10. An animal model, which comprises white zebrafish or transparent zebrafish as experimental animal.

11. The animal model of claim 10, which is used in disease model, aging investigation, drug screening, toxicant screening, embryo development or organ differentiation.

12. The animal model of claim 11, which is used in visual system model, laboratory model in comparative study, immunological model system, drug screening and validation, GATA transcription factor model, developmental model system, toxicological and transgenic research, developmental expression of thrombin, pharmacological animal model, host model system of microbial pathology, neurotoxicological model, model for myelopoiesis during embryogenesis, human disease model or genetic research model system.

13. The animal model of claim 10, wherein the white zebrafish is used in phenotype of body color or pigment investigation.

14. The animal model of claim 10, wherein the transparent zebrafish is used in observation of viscera.
